(19) United States
(12) Patent Application Publication
(10) Pub. No.: US 2025/0320275 A1
(43) Pub. Date: Oct. 16, 2025

(54) METHOD FOR PREPARING UNDENATURED COLLAGEN TYPE II FOR ALLEVIATING JOINT PAIN

(71) Applicant: Beijing Semnl Biotechnology Co., Ltd., Beijing (CN)

(72) Inventors: Haiyan Wang, Beijing (CN); Jingqi Liu, Beijing (CN); Aiqing Liu, Beijing (CN); Shuang Liu, Beijing (CN); Lige Liu, Beijing (CN); Pengcheng Li, Huaibei (CN)

(21) Appl. No.: 19/064,754

(22) Filed: Feb. 27, 2025

(30) Foreign Application Priority Data
Apr. 10, 2024 (CN) 202410428375.1

(51) Int. Cl. C07K 14/78; A61K 38/39; C07K 1/12; C07K 1/30; C12N 1/20; C12N 9/76; C12R 1/225; C12R 1/44

(52) U.S. Cl. CPC C07K 14/78; A61K 38/39; C07K 1/122; C07K 1/30; C12N 1/20; C12N 9/6427; C12Y 304/21004; C12R 2001/225; C12R 2001/44

(57) ABSTRACT

A method for preparing nondenatured collagen type II for alleviating joint pain includes pulverizing a cartilage raw material and defatting to obtain a total protein crude extract; adding anhydrous ethanol to the total protein crude extract, soaking, stirring, and centrifuging to obtain an anhydrous ethanol insoluble material; dissolving the anhydrous ethanol insoluble material in water, adjusting pH to acidic, stirring and centrifuging to obtain an acid-insoluble material; dissolving the acid-insoluble material in water, performing enzymatic hydrolysis and centrifuging to obtain an enzymatic hydrolysis precipitate; dissolving the enzymatic hydrolysis precipitate in water, followed by performing co-fermentation, and centrifuging to obtain a fermentation precipitate; and adding a water activity regulator to the fermentation precipitate, and then drying to obtain the nondenatured collagen type II. The content of protein, hydroxyproline and nondenatured collagen type II in the prepared nondenatured collagen type II is high, and the shelf life is prolonged.

### CROSS-REFERENCE TO RELATED APPLICATION

This application claims priority to Chinese Patent Application No. 202410428375.1, filed Apr. 10, 2024, which is herein incorporated by reference in its entirety.

### TECHNICAL FIELD

The disclosure relates to the field of biomedical technologies, and more particularly to a method for preparing undenatured collagen type II for alleviating joint pain.

### BACKGROUND

Researchers have currently discovered 28 different types of collagen in the human body, with collagen type I, type II, and type III being relatively abundant. Collagen type I and type III are primarily found in the skin, providing skin with tensile strength and elasticity, while collagen type II is mainly found in cartilage, maintaining its structure and function. Osteoarthritis is a degenerative joint disease that severely affects the quality of life of patients. In the joints of osteoarthritis patients, the structure of collagen type II undergoes changes, making it unable to maintain the normal structure of cartilage. Previously, common dietary supplements used to improve osteoarthritis included glucosamine, chondroitin sulfate, frankincense, turmeric, and fish oil. However, these dietary supplements have certain limitations. Nondenatured collagen type II, with advantages such as convenience, safety, non-toxicity, and antigen specificity, can be used for treating osteoarthritis. However, extracting nondenatured collagen type II from animal tissues still faces challenges such as low purity, high extraction difficulty, and high industrialization costs.

Chinese patent application with publication number of CN106916870A discloses a method for preparing a cartilage extract containing nondenatured collagen type II. The method includes defatting, disinfection, homogenization, enzymatic hydrolysis, filtration, and drying steps. The prepared cartilage extract contains 3.9% to 12.6% collagen type II, so the content of collagen type II still needs to be further improved. In addition, this patent application does not mention how to achieve stable storage of the cartilage extract. Chinese patent application with publication number of CN115120618A discloses a cartilage extract with immune response improvement effects, its preparation method, and its use. By adding sodium hyaluronate and lentinan in a weight ratio of 4:1 to 1:1 to the enzymatic hydrolysate, a cartilage extract containing an effective amount of nondenatured collagen type II for improving immune response is obtained. This extract can simultaneously prevent microbial spoilage and denaturation of collagen type II during storage, thus improving product safety and shelf stability.

Therefore, there is an urgent need to provide a method for preparing nondenatured collagen type II with high protein content, hydroxyproline content, and nondenatured collagen type II content, with a long shelf life.

### SUMMARY

The disclosure addresses the problems existing in the prior art and provides a method for preparing nondenatured collagen type II for alleviating joint pain. The method involves defatting, alcohol extraction, acid extraction, enzymatic hydrolysis, and the addition of a water activity regulator to a cartilage raw material to obtain the nondenatured collagen type II. The protein content, hydroxyproline content, and nondenatured collagen type II content obtained using this method are all relatively high, while also extending the product's shelf life.

To achieve the above objectives, the technical solution adopted by the disclosure is as follows.

A first aspect of the disclosure lies in providing a method for preparing nondenatured collagen type II for alleviating joint pain. The method includes the following steps S1 to S6:
S1: pulverizing a cartilage raw material to obtain a pulverized cartilage raw material, and defatting the pulverized cartilage raw material to obtain a total protein crude extract;
S2: adding anhydrous ethanol to the total protein crude extract, soaking and stirring the total protein crude extract in the anhydrous ethanol to obtain a first mixture, and centrifuging the first mixture to obtain an anhydrous ethanol insoluble material;
S3: dissolving the anhydrous ethanol insoluble material in water to obtain a second mixture, adding a potential of Hydrogen (pH) regulator into the second mixture to adjust pH to acidic, stirring the second mixture added with the pH regulator to obtain a third mixture, and centrifuging the third mixture to obtain an acid-insoluble material;
S4: dissolving the acid-insoluble material in water to obtain a fourth mixture, performing enzymatic hydrolysis on the fourth mixture using an enzyme preparation, followed by centrifuging to obtain an enzymatic hydrolysis precipitate;
S5: dissolving the enzymatic hydrolysis precipitate in water to obtain a fifth mixture, performing co-fermentation on the fifth mixture, followed by centrifuging to obtain a fermentation precipitate; and
S6: adding a water activity regulator to the fermentation precipitate to obtain a sixth mixture, and drying the sixth mixture to obtain the nondenatured collagen type II.

Preferably, in step S1, the cartilage raw material is pulverized into cartilage particles of 0.5 cm to 2 cm, the cartilage particles are soaked in NaOH solution, and then taken out and washed with water to neutrality to obtain washed cartilage particles, the washed cartilage fragments are mixed with water according to a weight ratio of material to liquid of 1:5 to 40, and then subjected to ultrasonic treatment and crushing to obtain the total protein crude extract.

Further preferably, in step S1, the weight fraction of the NaOH solution is 0.1% to 5%, the weight ratio of the NaOH solution to the cartilage particles is 6 to 12:1, and the soaking time is 10 hours to 24 hours.

Further preferably, in step S1, the ultrasonic power is 150 Watts (W) to 400 W, the ultrasonic wind speed is 5 meters per second (m/s) to 500 m/s, the ultrasonic time is 10 minutes to 80 minutes, and the ultrasonic treatment is performed 1 time to 3 times.

Preferably, in step S1, the cartilage raw material is sourced from chicken, shark, sheep, cow, or pig.

Preferably, in step S2, the amount of the anhydrous ethanol added is 2-10 times the weight of the total protein crude extract.

Preferably, in step S2, the soaking and stirring time is 2 hours to 6 hours.

Preferably, in step S3, the anhydrous ethanol insoluble material is dissolved in 5-20 times its weight of purified water.

Preferably, in step S3, the pH regulator is HCl solution with a weight fraction of 5% to 30%, the pH is adjusted to 1.8-2.5, and the stirring time is 4 hours to 24 hours.

Further preferably, in step S3, the pH is adjusted to 1.8-2.2, and the stirring time is 12 hours to 18 hours.

Preferably, in step S4, the acid-insoluble material is dissolved in 5-20 times its weight of purified water.

Preferably, in step S4, before the enzymatic hydrolysis, the pH of the fourth mixture is adjusted to 7.5-8.5 using a pH regulator, the enzyme preparation is trypsin, and the addition amount of the enzyme preparation is 0.1% to 2% of the acid-insoluble material's weight. The enzymatic hydrolysis time is 2 hours to 10 hours, and the enzymatic hydrolysis temperature is 37° C.

Further preferably, in step S4, the pH regulator added before the enzymatic hydrolysis is at least one selected from the group consisting of NaOH, Na₂CO₃, NaHCO₃, K₂CO₃, KHCO₃, CaCO₃, and CaO.

Preferably, in step S5, the enzymatic hydrolysis precipitate is dissolved in 5-20 times its weight of purified water.

Preferably, in step S5, the fermentation is co-fermentation, and the fermentation process is specifically as follows:

F1: inoculating Lactobacillus bulgaricus into the fifth mixture, with an inoculum amount of 0.1% to 3% by weight of the fifth mixture, fermenting the fifth mixture inoculated with the Lactobacillus bulgaricus for 4 hours to 18 hours at 40° C. to 43° C., followed by centrifuging to obtain an initial fermentation precipitate; and

F2: adding water to the initial fermentation precipitate to obtain a seventh mixture, inoculating Staphylococcus carnosus or Staphylococcus xylosus into the seventh mixture, with an inoculum amount of 0.5% to 5% by weight of the seventh mixture, fermenting the seventh mixture inoculated with the Staphylococcus carnosus or the Staphylococcus xylosus for 2 hours to 8 hours at 30° C. to 35° C., followed by centrifuging to obtain the fermentation precipitate.

Further preferably, in step F2, the amount of the water added is 5 times to 20 times the weight of the initial fermentation precipitate.

Further preferably, post-processing of the fermentation precipitate is performed as follows: adding 5-10 times the weight of purified water to the fermentation precipitate, adjusting the pH to 1.5-2, allowing it to stand for 2-6 hours, and centrifuging to obtain a first precipitate; then, adding 5-10 times the weight of purified water to the first precipitate, adjusting the pH to 1.5-2, allowing it to stand for 2-6 hours, and centrifuging to obtain a second precipitate; and finally, washing the second precipitate with purified water 2-4 times.

Preferably, in step S6, a weight ratio of the water activity regulator to the fermentation precipitate is 1:0.1 to 5, more preferably 1:0.2 to 2.

Preferably, in step S6, the water activity regulator is at least one selected from the group consisting of mannitol, goji berry polysaccharides, and guar gum.

Further preferably, the water activity regulator includes mannitol, goji berry polysaccharides, and guar gum.

Even further preferably, the weight ratio of mannitol, goji berry polysaccharides, and guar gum is 1:2-10:1-8, more preferably 1:2-5:2-5.

Preferably, in step S6, the drying is performed using forced air drying below 45° C. or freeze drying.

Compared to the prior art, the disclosure has the following beneficial effects: The preparation method of the disclosure involves a combination of processes such as defatting, ethanol extraction, acid extraction, enzymatic hydrolysis by protease, microbial fermentation, and the addition of the water activity regulator, which work together synergistically. In particular, by selecting the specific enzyme preparation, the fermentation agent, and the water activity regulator, the nondenatured collagen type II prepared in this way has a high content of protein, hydroxyproline, and nondenatured collagen type II. The protein content is over 30%, the hydroxyproline content is over 3.0%, and the nondenatured collagen type II content is over 18%. At the same time, the shelf life is extended. The disclosure reduces production costs, is suitable for industrial production, and the resulting nondenatured collagen type II can be used as the raw material for pharmaceuticals, cosmetics, health foods, and food products to improve and treat osteoarthritis.

### DETAILED DESCRIPTION OF EMBODIMENTS

It is worth noting that the raw materials used in the disclosure are all commercially available products, and their sources are not specifically limited.

The following raw materials are provided for exemplary purposes:

Trypsin and alkaline protease are purchased from Nanning Pangbo Biotechnology Co., Ltd. Staphylococcus xylosus and Staphylococcus carnosus are purchased from Shandong Pingju Biotechnology Co., Ltd. Mannitol, goji berry polysaccharides, and guar gum are purchased from Shandong Siyang Biotechnology Co., Ltd. Nondenatured collagen type II content testing kits are purchased from Shanghai Lianzu Biotechnology Co., Ltd. Lactobacillus bulgaricus is purchased from Guangdong Hongyou Biotechnology Co., Ltd.

### Embodiment 1

A method for preparing nondenatured collagen type II for alleviating joint pain includes the following steps.

S1:1000 grams (g) chicken breast cartilage is crushed into 0.5-2 centimeters (cm) particles, and the cartilage particles are soaked in 10 kilograms (kg) NaOH solution with a concentration of 0.5% for 12 hours, followed by washing with water to neutrality to obtain washed cartilage particles, the washed cartilage particles are mixed with water according to the ratio of material to liquid of 1:10, and then ultrasonicated twice at 200 W power and 150 m/s wind speed for 60 minutes each time to obtain a first mixture. The first mixture is defatted and centrifuged to obtain a total protein crude extract.

S2: the total protein crude extract is added into 10 liters (L) of absolute ethanol and stirred for 4 hours, followed by centrifuging to remove ethanol-soluble proteins, and evaporating the solvent to obtain a precipitate.

S3: the precipitate from step S2 is added into 10 kg purified water, followed by adjusting pH to 2.2, stirring for 18 hours, centrifuging to remove acid-soluble proteins, thereby obtaining the acid-insoluble material.

S4: the purified water is added to the acid-insoluble material from step S3 to obtain a second mixture, the addition amount of the purified water is 10 times of the weight of acid-insoluble material, the pH of the second mixture is adjusted to 8, and then the trypsin is added into the second mixture to obtain a third mixture, the addition amount of the trypsin is 2% of the weight of the acid-insoluble material, the enzymatic hydrolysis is performed on the third mixture at 37° C. for 6 hours, followed by centrifuging to obtain the enzymatic hydrolysis precipitate.

S5: the purified water is added to the enzymatic hydrolysis precipitate to obtain a fourth mixture, the addition amount of the purified water is 10 times of the weight of the enzymatic hydrolysis precipitate, then Lactobacillus bulgaricus with 1% solution weight is inoculated into the fourth mixture at 40° C. and fermented for 6 hours, followed by centrifuging to obtain the first precipitate. The purified water is added into the first precipitate to obtain a fifth mixture, the addition amount of the purified water is 10 times of the weight of the first precipitate, Staphylococcus carnosus with 1% solution weight is inoculated into the fifth mixture at 35° C. and fermented for 8 hours, followed by centrifuging to obtain the second precipitate. The purified water is added into the second precipitate to obtain a sixth mixture, the addition amount of the purified water is 10 times of the weight of the second precipitate, the pH of the sixth mixture is adjusted to 1.5, followed by standing for 4 hours and then centrifuging to obtain the third precipitate, adjusting pH to 1.5 again, standing for 6 hours, washing with purified water 3 times, then centrifuging to obtain the fermented precipitate.

S6:100 g mannitol, 100 g goji berry polysaccharides, and 200 g guar gum are mixed to form a solution. The solution is added to the fermented precipitate and stirred thoroughly, followed by freeze-drying at −30° C. to obtain the nondenatured collagen type II product that relieves joint pain.

### Embodiment 2

A method for preparing nondenatured collagen type II for alleviating joint pain, differs from Embodiment 1 in that, in step S5, the Staphylococcus carnosus is replaced with Staphylococcus xylosus, and all other steps are the same as in Embodiment 1.

### Embodiment 3

A method for preparing nondenatured collagen type II for alleviating joint pain, differs from Embodiment 1 in that, in step S6, the freeze-drying is replaced with air drying at 30° C., and all other steps are the same as in Embodiment 1.

### Comparative Example 1

A method for preparing nondenatured collagen type II for alleviating joint pain, differs from Embodiment 1 in that, in step S4, the trypsin is replaced with alkaline protease, and all other steps are the same as in Embodiment 1.

### Comparative Example 2

A method for preparing nondenatured collagen type II for alleviating joint pain, differs from Embodiment 1 in that, in step S5, only Lactobacillus bulgaricus is inoculated in step S5. Specifically, in step S5: adding the purified water to the enzymatic hydrolysis precipitate with the addition amount of 10 times by weight of the enzymatic hydrolysis precipitate, inoculating Lactobacillus bulgaricus with 1% solution weight at 40° C. and fermenting for 6 hours, centrifuging to obtain the first precipitate, adding the purified water to the first precipitate with the addition amount of 10 times by weight of the first precipitate, adjusting the pH to 1.5, allowing to stand for 4 hours, centrifuging to obtain the second precipitate, adjusting the pH to 1.5 again, allowing to stand for 6 hours, and washing three times with purified water and then centrifuging to obtain the fermentation precipitate. All other steps are the same as in Embodiment 1.

### Comparative Example 3

A method for preparing nondenatured collagen type II for alleviating joint pain, differs from Embodiment 1 in that, only Staphylococcus xylosus is inoculated in step S5. Specifically, in step S5: adding the purified water to the enzymatic hydrolysis precipitate with the addition amount of 10 times by weight of the enzymatic hydrolysis precipitate, inoculating Staphylococcus xylosus with 1% solution weight at 35° C. and fermenting for 8 hours, centrifuging to obtain the first precipitate, adding the purified water to the first precipitate with the addition amount of 10 times by weight of the first precipitate, adjusting the pH to 1.5, allowing to stand for 4 hours, centrifuging to obtain the second precipitate, adjusting the pH to 1.5 again, allowing to stand for 6 hours, and washing three times with purified water and then centrifuging to obtain the fermentation precipitate. All other steps are the same as in Embodiment 1.

### Comparative Example 4

A method for preparing nondenatured collagen type II for alleviating joint pain, differs from Embodiment 1 in that, Lactobacillus plantarum and Staphylococcus vitulinus are inoculated in step S5. Specifically, in step S5: adding the purified water to the enzymatic hydrolysis precipitate with the addition amount of 10 times by weight of the enzymatic hydrolysis precipitate, inoculating Lactobacillus plantarum with 1% solution weight at 40° C. and fermenting for 6 hours, centrifuging to obtain the first precipitate, adding the purified water to the first precipitate with the addition amount of 10 times by weight of the first precipitate, inoculating Staphylococcus vitulinus with 1% solution weight at 35° C. and fermenting for 8 hours, centrifuging to obtain the second precipitate, adding the purified water to the second precipitate with the addition amount of 10 times by weight of the second precipitate, adjusting the pH to 1.5, allowing to stand for 4 hours, centrifuging to obtain the third precipitate, adjusting the pH to 1.5 again, allowing to stand for 6 hours, and washing three times with purified water and then centrifuging to obtain the fermentation precipitate. All other steps are the same as in Embodiment 1.

### Comparative Example 5

A method for preparing nondenatured collagen type II for alleviating joint pain, differs from Embodiment 1 in that, in step S6, 100 g of mannitol, 100 g of goji berry polysaccharides, and 200 g of guar gum are replaced with 400 g of mannitol. All other steps are the same as in Embodiment 1.

### Comparative Example 6

A method for preparing nondenatured collagen type II for alleviating joint pain, differs from Embodiment 1 in that, in step S6, 100 g of mannitol, 100 g of goji berry polysaccharides, and 200 g of guar gum are replaced with 100 g of sorbitol, 100 g of tremella polysaccharides, and 200 g of carrageenan. All other steps are the same as in Embodiment 1.

### Experimental Example 1: Nondenatured Collagen Type II Indicators

The protein, hydroxyproline, and nondenatured collagen type II content were tested for Embodiments 1-3 and Comparative Examples 1-6. The protein content was measured according to the method for determining protein in food in the GB 5009.5-2016 National Food Safety Standard. The hydroxyproline content was measured according to the method for determining hydroxyproline in meat and meat products in GB/T 9695.23-2008. The nondenatured collagen type II content was detected using a type II collagen assay kit.

TABLE 1
indicator content in nondenatured collagen
type II products prepared by the disclosure

| | | | Nondenatured |
|---|---|---|---|
| | Protein | Hydroxyproline | collagen type II |
| Group | content (%) | content (%) | content (%) |
| Embodiment 1 | 34.7 | 3.34 | 21.65 |
| Embodiment 2 | 32.5 | 3.45 | 22.70 |
| Embodiment 3 | 31.9 | 3.29 | 18.32 |
| Comparative | 42.8 | 3.58 | 3.98 |
| Example 1 | | | |
| Comparative | 38.9 | 3.22 | 5.01 |
| Example 2 | | | |
| Comparative | 37.6 | 3.12 | 6.87 |
| Example 3 | | | |
| Comparative | 34.1 | 2.98 | 5.71 |
| Example 4 | | | |
| Comparative | 32.8 | 3.21 | 21.54 |
| Example 5 | | | |
| Comparative | 33.5 | 3.14 | 20.06 |
| Example 6 | | | |

Hydroxyproline is a characteristic amino acid of collagen, representing the total collagen content. The content of nondenatured collagen type II indicates the amount of active ingredients in the product. From the experimental results, it can be seen that in Embodiments 1-3 of the disclosure, the protein content is above 30%, the hydroxyproline content is above 3.0%, and the nondenatured collagen type II content is above 18%. Although the protein content in Embodiments 1-3 is slightly lower than in Comparative Examples 1-4, the nondenatured collagen type II content is significantly higher in the disclosed embodiments. Therefore, the method of the disclosure effectively removes impurity proteins and increases the content of nondenatured collagen type II.

### Experimental Example 2: Product Moisture and Microbial Indicators During Shelf Life

Since nondenatured collagen type II is prone to inactivation and denaturation at high temperatures, high-temperature sterilization was not used during the production process. Instead, high-acid sterilization was employed, and the water activity regulator was added to inhibit microbial growth during the shelf life. The moisture and microbial indicators of Embodiments 1, 3 and Comparative Examples 5-6 were tested during the shelf life. The moisture content was tested according to GB 5009.3-2016, the moisture activity was tested according to GB 5009.238-2016, and the colony forming unit was determined according to GB 4789.2-2022. The product is considered qualified if the moisture content is ≤10%, the moisture activity is ≤0.8, and the colony forming unit is ≤3000 colony forming units per gram (cfu/g). The testing results are shown in Table 2.

TABLE 2
moisture and microbial testing results

| | | | | Comparative | Comparative |
|---|---|---|---|---|---|
| Group Indictor | | Embodiment 1 | Embodiment 3 | Example 5 | Example 6 |
| 0 | Moisture | 6.72 | 4.54 | 6.31 | 6.23 |
| | content (%) | | | | |
| | Moisture | 0.42 | 0.38 | 0.78 | 0.74 |
| | activity (AW) | | | | |
| | Colony forming | 120 | 140 | 160 | 100 |
| | unit (cfu/g) | | | | |
| 6 | Moisture | 6.74 | 4.66 | 7.35 | 7.28 |
| mouths | content (%) | | | | |
| | Moisture | 0.42 | 0.38 | 0.82 | 0.76 |
| | activity (AW) | | | | |
| | Colony forming | 140 | 140 | 1400 | 800 |
| | unit (cfu/g) | | | | |
| 12 | Moisture | 6.87 | 4.80 | 7.89 | 7.65 |
| mouths | content (%) | | | | |
| | Moisture | 0.45 | 0.41 | 0.88 | 0.78 |
| | activity (AW) | | | | |
| | Colony forming | 160 | 140 | 3200 | 1400 |
| | unit (cfu/g) | | | | |
| 18 | Moisture | 6.91 | 4.80 | 8.06 | 7.87 |
| mouths | content (%) | | | | |
| | Moisture | 0.45 | 0.41 | 0.87 | 0.80 |
| | activity (AW) | | | | |
| | Colony forming | 160 | 140 | 4600 | 2600 |
| | unit (cfu/g) | | | | |
| 24 | Moisture | 7.12 | 5.14 | 8.17 | 8.06 |
| mouths | content (%) | | | | |
| | Moisture | 0.45 | 0.41 | 0.91 | 0.81 |
| | activity (AW) | | | | |
| | Colony forming | 200 | 210 | 1.5*10⁴ | 5900 |
| | unit (cfu/g) | | | | |
| 30 | Moisture | 7.13 | 5.11 | 8.22 | 8.17 |
| mouths | content (%) | | | | |
| | Moisture | 0.45 | 0.41 | 0.93 | 0.84 |
| | activity (AW) | | | | |
| | Colony forming | 210 | 210 | 3.4*10⁴ | 1.3*10⁴ |
| | unit (cfu/g) | | | | |
| 36 | Moisture | 7.13 | 5.11 | 8.29 | 8.12 |
| mouths | content (%) | | | | |
| | Moisture | 0.45 | 0.41 | 0.94 | 0.88 |
| | activity (AW) | | | | |
| | Colony forming | 210 | 210 | 1.2*10⁵ | 3.5*10⁵ |
| | unit (cfu/g) | | | | |

From Table 2, it can be seen that the products of the disclosure in Embodiments 1 and 3 meet the moisture and microbial standards during the shelf life, while the products in Comparative Examples 5-6 exceed the specified limits for moisture activity and microorganisms during the shelf life.

### Experimental Example 3

30 patients with osteoarthritis were recruited, and a double-blind randomized controlled experiment was conducted. The Western Ontario and McMaster Universities Index (WOMAC) score was used to compare the effects of oral administration of nondenatured collagen type II produced according to Embodiment 1 and a placebo (maltodextrin) over a period of 3 months.

The results are shown in Table 3.

TABLE 3
WOMAC score results

| | Nondenatured collagen type II group | | | Placebo group | | |
|---|---|---|---|---|---|---|
| | Experiment | After 3 | | Experiment | After 3 | |
| Score | beginning | mouths | P value | beginning | mouths | P value |
| Pain | 7.34 ± 1.73 | 2.01 ± 0.41 | P < 0.05^{&#} | 7.21 ± 1.87 | 7.95 ± 1.62 | P > 0.05 |
| Stiffness | 6.87 ± 1.18 | 3.25 ± 0.52 | P < 0.05^{&#} | 6.53 ± 1.64 | 7.46 ± 1.26 | P > 0.05 |
| Difficulty | 8.45 ± 2.51 | 3.18 ± 0.67 | P < 0.05^{&#} | 8.27 ± 2.07 | 8.60 ± 2.86 | P > 0.05 |
| of daily | | | | | | |
| life | | | | | | |

Note: & denotes intra-group comparison; # denotes comparison with the control group.

The nondenatured collagen type II prepared using the method of the disclosure can significantly alleviate pain and stiffness, and reduce the difficulty in daily life for patients with osteoarthritis. It has a notable effect on improving and treating osteoarthritis.

Finally, it should be noted that the above content is provided for illustrating the technical solution of the disclosure and does not limit the scope of the disclosure. Any simple modifications or equivalent substitutions made by those skilled in the art based on the technical solution of the disclosure shall still fall within the spirit and scope of the disclosure.

1. A method for preparing undenatured collagen type II for alleviating joint pain, comprising:
S1, pulverizing a cartilage raw material to obtain a pulverized cartilage raw material, and defatting the pulverized cartilage raw material to obtain a total protein crude extract;
S2, adding anhydrous ethanol to the total protein crude extract, soaking and stirring the total protein crude extract in the anhydrous ethanol to obtain a first mixture, and centrifuging the first mixture to obtain an anhydrous ethanol insoluble material;
S3, dissolving the anhydrous ethanol insoluble material in water to obtain a second mixture, adding a pH regulator into the second mixture to adjust pH to acidic, stirring the second mixture added with the pH regulator to obtain a third mixture, and centrifuging the third mixture to obtain an acid-insoluble material;
S4, dissolving the acid-insoluble material in water to obtain a fourth mixture, performing enzymatic hydrolysis on the fourth mixture using an enzyme preparation, followed by centrifuging to obtain an enzymatic hydrolysis precipitate;
S5, dissolving the enzymatic hydrolysis precipitate in water to obtain a fifth mixture, performing co-fermentation on the fifth mixture, followed by centrifuging to obtain a fermentation precipitate; and
S6, adding a water activity regulator to the fermentation precipitate to obtain a sixth mixture, and drying the sixth mixture to obtain the undenatured collagen type II;
wherein before the enzymatic hydrolysis, step S4 further comprises: adding a pH regulator into the fourth mixture to adjust pH to 7.5-8.5; and the enzyme preparation is trypsin, an addition amount of the enzyme preparation is 0.1% to 2% of a weight of the acid-insoluble material, time for the enzymatic hydrolysis is 2 hours to 10 hours, and a temperature for the enzymatic hydrolysis is 37° C.;
wherein in step S5, the co-fermentation comprises:
F1: inoculating Lactobacillus bulgaricus into the fifth mixture, wherein an inoculum amount of the Lactobacillus bulgaricus is 0.1% to 3% of a weight of the fifth mixture; and fermenting the fifth mixture inoculated with the Lactobacillus bulgaricus for 4 hours to 18 hours at 40° C. to 43° C., followed by centrifuging to obtain an initial fermentation precipitate; and
F2: adding water to the initial fermentation precipitate to obtain a seventh mixture, and inoculating Staphylococcus carnosus or Staphylococcus xylosus into the seventh mixture, wherein an inoculum amount of the Staphylococcus carnosus or the Staphylococcus xylosus is 0.5% to 5% of a weight of the seventh mixture; and fermenting the seventh mixture inoculated with the Staphylococcus carnosus or the Staphylococcus xylosus for 2 hours to 8 hours at 30° C. to 35° C., followed by centrifuging to obtain the fermentation precipitate; and
wherein in step S6, the water activity regulator comprises mannitol, Lycium barbarum polysaccharides, and guar gum, a weight ratio of the water activity regulator to the fermentation precipitate is 1:0.1 to 5, and a weight ratio of the mannitol, the Lycium barbarum polysaccharides, and the guar gum is 1:2 to 10:1 to 8.

2. The method according to claim 1, wherein step S1 specifically comprises:
pulverizing the cartilage raw material into cartilage particles of 0.5 cm to 2 cm, soaking the cartilage particles in NaOH solution, taking out the cartilage particles from the NaOH solution and washing the cartilage particles with water to neutralize to obtain washed cartilage particles, and mixing the washed cartilage particles with water according to a weight ratio of material to liquid of 1:5 to 40, followed by performing ultrasonic treatment and crushing to obtain the total protein crude extract.

3. The method according to claim 2, wherein in step S1, a weight fraction of the NaOH solution is 0.1% to 5%, time for the soaking is 10 hours to 24 hours, ultrasonic power is 150 W to 400 W, an ultrasound velocity is 5 m/s to 500 m/s, ultrasonic time is 10 minutes to 80 minutes, and the ultrasonic treatment is performed 1 time to 3 times.

4. The method according to claim 1, wherein the cartilage raw material is sourced from one selected from the group consisting of chicken, shark, sheep, cow, and pig.

5. The method according to claim 1, wherein an amount of the anhydrous ethanol added in step S2 is 2-10 times of a weight of the cartilage raw material.

6. The method according to claim 1, wherein time for the soaking and stirring in step S2 is 2 hours to 6 hours.

7. The method according to claim 1, wherein in step S3, the pH regulator is an HCl solution with a weight fraction of 5% to 30%, the pH is adjusted to 1.8 to 2.5, and stirring time is 4 hours to 24 hours.

8. The method according to claim 7, wherein in step S3, the pH is 1.8 to 2.2, and the stirring time is 12 hours to 18 hours.

9. The method according to claim 1, wherein in step F2, an amount of the water added is 5-20 times of a weight of the initial fermentation precipitate.

10. The method according to claim 1, wherein the method further comprises a post-treatment process for the fermentation precipitate obtained in step S5, specifically:
adding purified water to the fermentation precipitate to obtain an eighth mixture, wherein an addition amount of the purified water is 5-10 times of a weight of the fermentation precipitate, adjusting pH of the eighth mixture to 1.5 to 2, followed by standing for 2 hours to 6 hours, and then centrifuging to obtain a first precipitate; and adding purified water to the first precipitate to obtain a ninth mixture, wherein an addition amount of the purified water is 5-10 times of a weight of the first precipitate, adjusting pH of the ninth mixture to 1.5 to 2, followed by standing for 2 hours to 6 hours, and then centrifuging to obtain a second precipitate, and washing the second precipitate with purified water 2 times to 4 times.
